(19) United States
(12) Patent Application Publication
(10) Pub. No.: US 2025/0375153 A1
(43) Pub. Date: Dec. 11, 2025

(54) IMAGING APPARATUS AND METHOD FOR DETERMINING AN IMAGING PROTOCOL FOR A SUB-AREA

(71) Applicant: Siemens Healthineers AG, Forchheim (DE)

(72) Inventors: Dominik RUPP, Tuchenbach (DE); Miriam VAN DE STADT-LAGEMAAT, Erlangen (DE); Frank MEHLER, Neunkirchen am Brand (DE)

(73) Assignee: Siemens Healthineers AG, Forchheim (DE)

(21) Appl. No.: 19/233,143

(22) Filed: Jun. 10, 2025

(30) Foreign Application Priority Data
Jun. 11, 2024 (EP) 24181396.3

(51) Int. Cl. A61B 5/00; A61B 5/055; G01R 33/54

(52) U.S. Cl. CPC A61B 5/4547; A61B 5/055; G01R 33/543

(57) ABSTRACT

A computer-implemented method comprises: acquiring information about a region of a body of a patient; provisioning an input option for acquiring information about a sub-area of the region of the body as a function of the information about the region of the body; acquiring a diagnostically relevant sub-area as a function of the input option for acquiring the information about the sub-area of the region of the body; establishing the imaging protocol for acquiring image data of the diagnostically relevant sub-area of the region of the body of the patient as a function of the diagnostically relevant sub-area; provisioning the imaging protocol for acquiring image data of the diagnostically relevant sub-area of the region of the body via the imaging apparatus. The input option includes a plurality of sub-areas of the region of the body.

### CROSS-REFERENCE TO RELATED APPLICATION(S)

The present application claims priority under 35 U.S.C. § 119 to European Patent Application No. 24181396.3, filed Jun. 11, 2024, the entire contents of which are incorporated herein by reference.

### FIELD

One or more example embodiments of the present invention relate to a method for determining an imaging protocol for an imaging examination via an imaging apparatus. Moreover, one or more example embodiments of the present invention relate to a medical imaging apparatus that is suitable for recording image data of a region of the body of a patient.

### BACKGROUND

The planning of an imaging protocol for acquisition of image data of a diagnostically relevant region of the body of a patient via an imaging apparatus can depend on patient-specific prerequisites and/or on user-specific inputs during a patient registration and also on a preparation of the patient for an imaging examination. In the planning of the imaging protocol for example a laterality of the diagnostically relevant region of the body (for example the presence of a left or right half of the body), a division of the region of the body into relevant and irrelevant sub-areas and/or an orientation of the region of the body in a reference system are to be noted. For imaging protocols for the tooth or jaw region of the patient in particular, a specification of a suitable imaging area, as well as a choice of relevant areas of region of the body due to the symmetrical structure of the dental arches (for example left, right, or frontal lower jaw and/or upper jaw) and the undulating shape of the dental arches can be complex. Therefore effort is involved in training users of the imaging apparatus or said users must have specialized knowledge and empirical values available to them in order to guarantee an error-free acquisition of image data of the diagnostically relevant regions of the body via an imaging apparatus under everyday clinical conditions.

Commercially-available imaging apparatuses typically have a planning program for planning of an imaging protocol for an imaging examination of a region of the body of a patient. However such planning programs seldom take account of the differentiability of the region of the body per se, such as for example a laterality and/or subdivision of the region of the body. Therefore for example an imaging can be prepared by a planning program for the entire hip region of a patient, although only the right hip of the patient is diagnostically relevant. For a proportion of examinations such a rough choice of a diagnostically relevant region of the body is sufficient, depending on the complexity of the diagnosis setting or the course of the examination, as a rule however furthermore detailed examinations must be added.

In a few application cases various parameters or scenarios are preset for the user by the planning program, wherein the user must be familiar with the planning program however, in particular when it needs to be adapted, and has to have completed appropriate training. What is more, the complexity of the imaging planning leads in part to an inefficient or time-intensive determination of the image protocol, for example by a duplication of instructions, guidance, workflows, which can ideally be avoided. In other application cases an (almost complete) manual creation of an imaging protocol by a user is needed, in particular when only a specific area of a region of the body, for example the pubic bone region of a hip region of the patient, is to be recorded by an imaging examination.

An object of one or more example embodiments of the present invention is to improve the efficiency of a definition of an imaging protocol for an acquisition of image data of a region of the body of a patient via an imaging apparatus. At least this object is achieved by the features of the independent claims. Advantageous embodiments and expedient developments are described in the dependent claims.

The inventive computer-implemented method for determining an imaging protocol for acquisition of image data of a diagnostically relevant sub-area of a region of the body of a patient via an imaging apparatus comprises the following steps: acquisition of information about the region of the body, provision of an input option for acquisition of information about a sub-area of the region of the body as a function of the information about the region of the body, wherein the input option comprises a plurality of sub-areas of the region of the body, acquisition of the diagnostically relevant sub-area as a function of the input option for acquisition of the information about the sub-area of the region of the body, determination of the imaging protocol for acquisition of image data of the diagnostically relevant sub-areas of the region of the body of the patient as a function of the diagnostically relevant sub-areas acquired, provision of the imaging protocol for acquisition of image data of the diagnostically relevant sub-area of the region of the body via the imaging apparatus.

The acquisition of image data of the diagnostically relevant sub-area of the region of the body of the patient via an imaging apparatus can also be referred to as an imaging examination.

The method can preferably comprise an acquisition of image data of the diagnostically relevant sub-area of the region of the body of the patient via the imaging protocol provided. In particular the method can comprise carrying out an imaging examination as a function of the imaging protocol. In other words the imaging examination can be controlled at the imaging apparatus by the imaging protocol. The imaging examination can comprise carrying out one or more imaging sequences.

An imaging protocol can in particular comprise one or more imaging parameters and/or imaging parameter groups. Examples of imaging parameters are a spatial resolution, a contrast, a slice thickness, a dimension of an imaging volume, a relaxation time, an echo time and the like. An imaging parameter can in particular comprise any given image-relevant setting of the imaging apparatus, but also a parameter of a workflow of the imaging examination. Furthermore, parameters that define an imaging area of the imaging apparatus can also be understood as imaging parameters. An imaging volume can for example represent a volume with a highest homogeneity of a magnetic field, in particular an isocenter, of the magnetic resonance apparatus.

The region of the body can comprise a joint, a bone, a group of bones, a part of the body, an extremity or the like. For example the region of the body can be a hip region, a shoulder region, a knee region, a cerebral region, an eye region or any other anatomical structure. Furthermore the region of the body can comprise a tissue structure or an organ, such as for example a heart, a liver, a kidney, or the like. The region of the body can also be referred to as an area of the body, a sub-area of a body, a section of the body or part of the body. Preferably the region of the body of the patient comprises a jaw region or a tooth region.

The region of the body in particular comprises a plurality of sub-areas. A sub-area of the region of the body can also be referred to as subregion, part section or subsection. A sub-area preferably comprises a part area of the region of the body. A sub-area can in particular represent a section of the region of the body, which is distinguished or delimited because of tissue properties and/or an anatomical structure from further sections or sub-areas of the region of the body. For example individual elements of a finger or of a hand bone can represent sub-areas of the region of the body of the patient.

An alignment, a location and/or an extent of a first sub-area of the region of the body can further deviate from an alignment and/or extent of a second sub-area of the region of the body. It can be necessary or desirable to define different imaging areas as a function of the alignment, location and/or extent of the first sub-area and of the second sub-area of the region of the body. Through a restriction or adaptation of the imaging areas to a plurality of sub-areas of the region of the body, a duration of an imaging examination of the sub-areas can be reduced in an advantageous way. If the region of the body is the jaw region of a patient, a sub-area of the region of the body can for example be a section of the lower jawbone (corpus mandibulae) and/or the area of the jaw angle (angulus mandibulae). A further example of sub-areas of a region of the body can be teeth and/or sections of a dental arch. The sub-areas of the region of the body can adjoin one another, be arranged next to one another and/or overlap with and/or include one another.

A sub-area of a region of the body, which is especially relevant for an examination task, set of findings, treatment or diagnosis, can be referred to as a diagnostically relevant sub-area. In other words the diagnostically relevant sub-area refers to one or more selected sub-areas and/or to the area of the region of the body of a patient for which an imaging examination is to be carried out. The diagnostically relevant sub-area can in particular represent a region of interest. It is conceivable for the diagnostically relevant sub-area to correspond to one or more sub-areas of the region of the body of the patient as regards a property and/or extent. The diagnostically relevant sub-area can in particular comprise a plurality of sub-areas of the region of the body of the patient. The diagnostically relevant sub-area of the region of the body of the patient is in particular a sub-area selected by the user from a plurality of sub-areas of a region of the body provided.

Image data can in particular represent data that is acquired via the imaging apparatus from the region of the body and/or a sub-area of the patient. Image data can in particular be an imaging examination via the imaging apparatus. Image data can in particular comprise both raw data and also images that are derived from the raw data. For example the image data can comprise digitized magnetic resonance signals acquired by a magnetic resonance apparatus. The image data can in particular be stored as complex values in a k-space matrix. Preferably however the image data also comprises magnetic resonance images that have been reconstructed as a function of the digitized magnetic resonance signals.

An imaging apparatus can in particular be an apparatus for acquisition of image data of an examination object, in particular of a region of the body or of one or more sub-areas of a region of the body of a patient. In other words an imaging examination is able to be carried out via the imaging apparatus. Alternative names for an imaging apparatus can be imaging device, medical imaging system or image acquisition unit. Preferably an imaging apparatus is embodied to record two-dimensional and/or three-dimensional image data, in particular time-dependent three-dimensional image data, of the examination object. Examples for imaging apparatuses are magnetic resonance devices, x-ray devices, computed tomography devices, Single-Photon Emission Computed Tomographs, Positron Emission Tomographs, but also mammography devices, ultrasound devices and the like. In a preferred form of embodiment the imaging apparatus is a magnetic resonance apparatus.

Information about the region of the body of the patient can comprise any given description of a type, of a designation, of a position and/or of an extent of the region of the body of the patient. The information about the region of the body can further comprise a selection and/or an identification of the region of the body or of an anatomical structure from a list or a database of regions of the body.

The information about the region of the body of the patient can be entered manually by a user of the imaging apparatus via a user interface. In particular the information about the region of the body can be selected via a selection of an anatomical structure or of a section of an anatomical structure as a function of a representation of the anatomical structure provided via the user interface by the user of the imaging apparatus. It is furthermore conceivable for the imaging apparatus to have a control unit and/or a processing unit, which are embodied to obtain the information about the region of the body of the patient as a function of patient information or via a suitable interface from a medical information system, a Cloud and/or a local memory unit. In particular the information about the region of the body can be determined via an algorithm or an image processing algorithm as a function of the patient data, image data of a previous imaging examination of the region of the body of the patient and/or an examination instruction.

It is conceivable for the information about the region of the body to be predefined. In this case the acquisition of the information about the region of the body can be undertaken in an automated manner or comprise a one-off definition of the information about the region of the body. For example an imaging apparatus can be embodied or employed just for the recording and/or acquisition of image data of jaw regions of patients. In particular the imaging apparatus can be embodied as a dedicated scanner, for example an extremity scanner or a dental scanner. In one form of embodiment the information about the region of the body is initially acquired (for example in a configuration step of the imaging apparatus in a medical facility) and is subsequently stored for one or more following executions of the inventive method or determination of the imaging protocol for acquisition of image data of the diagnostically relevant sub-area of the region of the body of the patient as a function of the diagnostically relevant sub-area acquired.

The provision of the input option for acquisition of information about a sub-area of the region of the body preferably comprises output of the input option via an output unit and/or a user interface of the imaging apparatus. The acquisition of the information about the region of the body can also be comprised by the step of provision of the input option for acquisition of information about a sub-area of the region of the body. For example a user can first select a region of the body via a graphical representation of the regions of the body of the patient and immediately thereafter can be provided with the graphical representation of the region of the body.

Preferably the input option for acquisition of the information about the sub-area of the region of the body comprises a graphical representation of the region of the body and/or of one or more sub-areas of the region of the body of the patient. An input option can make it possible for a user to select options output, in particular sub-areas of the region of the body. In particular the input option can be embodied for selection of one or more sub-areas of the region of the body of the patient. Preferably the input option comprises an output of information about a sub-area of the region of the body.

The diagnostically relevant sub-area of the region of the body of the patient can in particular represent a sub-area selected by the user from a plurality of sub-areas of a region of the body provided, which are provided via the input option for acquisition of the information about the sub-area of the region of the body.

For example the input option for acquisition of the information about the sub-area of the region of the body for selection of one or more sub-areas and/or adaptation of a parameter of an imaging protocol of the diagnostically relevant sub-area can be output to the user via a graphical user interface, in particular a monitor or a touchscreen. The input option can for example comprise one or more input masks, which make it possible for the user to select a sub-area, to determine one or more imaging areas, to adapt or to change one or more parameters of a diagnostically relevant sub-area and/or of one or more imaging areas.

An imaging area can represent a common imaging area. A common imaging area can comprise one or more diagnostically relevant sub-area of the region of the body of the patient. The input option for acquisition of the information about the sub-area of the region of the body can preferably, in particular with a choice of a plurality of sub-areas, comprise a graphical representation of the common imaging area, in order to illustrate to the user the common imaging area of the diagnostically relevant sub-areas of the region of the body. On the basis of this information the user can preferably be prompted to make an ideal choice of diagnostically relevant sub-areas.

In one example the information about the region of the body of the patient comprises a designation or an identification of a section of an anatomical structure, in particular of a section of a tooth or jaw region of a patient, for example of a section of an upper jaw (maxilla). The input option for acquisition of the information of the sub-area can accordingly be provided to the user of the imaging apparatus as a function of the information about a region of the body via an output unit or a graphical user interface. For example the input option for acquisition of the information about the sub-area can be selected or retrieved from a database of a memory unit, which comprises a plurality of input options for selection of sub-areas for different regions of the body. In particular a processing unit and/or an algorithm, as a function of the information about a region of the body, can determine the input option in the form of a listing of sub-areas and/or a graphical representation and/or select it from a database. For example the input option for an upper jaw region of a patient can comprise a choice of (bone) sub-areas, such as the frontal process (processus frontalis), alveolar process (processus alveolaris) or the maxillary tuberosity (tuber maxillae).

A user of the imaging apparatus can for example be a medical specialist, in particular a dentist, a medical assistant or a member of medical staff of a practice or of a clinical facility. The user can be positioned or located at a site of the imaging apparatus, but also at any other given location. For example the user can be in another town, another region and/or another country and interact with the imaging apparatus or be remotely controlling said apparatus.

The acquisition of the diagnostically relevant sub-area of the region of the body can comprise an acquisition of an input via the input option for acquisition of the information about the sub-area of the region of the body. The acquisition of the diagnostically relevant sub-area in particular comprises the choice and/or identification of one or more sub-areas as diagnostically relevant sub-areas by an input of the user via the input option. In other words the acquisition of the diagnostically relevant sub-area can represent an acquisition or input of the user as a response by the user to the input option. The input of the user can thus comprise the information about the sub-area of the region of the body. The input of the user can in particular comprise one or more sub-areas of the region of the body.

Preferably the input option for acquisition of the information about the sub-area of the region of the body can be provided via an input interface or a user interface of the imaging apparatus. For example the input interface or the user interface can comprise a mouse, a keyboard, a touchscreen and/or a speech interface. The input of the user can preferably comprise a choice of a graphical object, which represents a sub-area of the region of the body of the patient. The input of the user can preferably comprise selection of a textual object, for example a selection of an element from a list. The input of the user can be made by a text-based input, for example by the input of an abbreviation, of an acronym or of a term for it into an input window, or a graphical input, for example a graphical selection via a cursor in a graphical input window. The text-based input can comprise one or more coordinates, one or more dimensions of the sub-area, a coordinate of a geometrical midpoint of the sub-area or the like. The input option provided by an input interface or a user interface of the imaging apparatus can provide the user with information, which can lead the user to the selection of a graphical object, selection of a textual object, of a text-based input and/or graphical input.

Preferably the diagnostically relevant sub-area corresponds to the sub-areas acquired as a function of the user's input. For example the diagnostically relevant sub-area can match the selection of one or more sub-areas by the user. Furthermore the diagnostically relevant sub-area can also comprise sub-areas of the region of the body that have not been acquired by a user as diagnostically relevant sub-areas. For example a sub-area identified by the user via an input option as diagnostically relevant can be supplemented by one or more sub-areas of the region of the body, which is necessary for the determination of the imaging protocol. If for example via the input option only the sub-areas of a tooth are acquired as a diagnostically relevant sub-area, the neighboring sub-areas can be added to the diagnostically relevant sub-area in order to enable the image data of the diagnostically relevant sub-areas to be acquired. The specifications or prerequisites for creation of the imaging protocol for specific sub-areas of regions of the body during the acquisition of the diagnostically relevant sub-area can be noted and/or included, in particular via a processing unit of the imaging apparatus.

The determination of the imaging protocol is undertaken as a function of the acquired diagnostically relevant sub-area. The provision of the image protocol can in particular be undertaken as a function of specifications or prerequisites for creation of the imaging protocol for specific sub-areas of regions of the body by the processing unit and/or an algorithm. The determination of the imaging protocol can in particular comprise at least one of the following steps: A selection of one or more imaging protocols, a creation of one or more imaging protocols, a selection of one or more imaging parameters, an adaptation of one or more imaging parameters, acquisition of an input of one or more imaging parameters by a user of the imaging apparatus. Preferably the determination of the imaging protocol is undertaken without further user interaction as a function of the diagnostically relevant sub-area. For example, with the aid of the input option for acquisition of the information about sub-area of the region of the body there can be an adaptation of a parameter of the imaging protocol and/or an adaptation of an imaging area via a processing unit of the imaging apparatus.

The provision of the imaging protocol can represent a final preparation step of an imaging examination. The imaging protocol can in particular be transferred by a processing unit of the imaging apparatus to a control unit of the imaging apparatus in order to carry out the imaging examination as a function of the imaging protocol.

In a preferred form of embodiment the provision of the input option for acquisition of information about a sub-area of the region of the body comprises an interactive option for selecting one or more diagnostically relevant sub-areas. The provision of the input option to a user is preferably undertaken via an output unit, in particular a monitor. The acquisition of the diagnostically relevant sub-area as a function of the input option preferably represents the only step and/or point in time in the inventive method for determining the imaging protocol that requires an active interaction with the user.

In an advantageous manner the inventive method can make possible an acquisition of information about a sub-area of a region of the body by a user of the imaging apparatus as a function of the input option provided. In an advantageous manner this enables a choice of the diagnostically relevant sub-area of the region of the body to be made taking into account both specifications or prerequisites for creation of the imaging protocol for specific sub-areas of regions of the body and/or regions of the body and also individual requirements of an anatomical structure of the region of the body of the patient.

Regions of the body of patient can differ significantly from one another individually. For example influencing variables such as a size, a gender and/or body circumference of a patient can influence a location of specific regions of the body or of a sub-area of a region of the body. Anatomical structures of the region of the body of the patient can however, for example due to a history of illness, also have entirely different shapes, dimensions or spatial alignments. In particular anatomical structures that are able to be subdivided into a number of almost similar or symmetrical sub-areas, such as for example a hand or a tooth region of a patient, require an intensive training of users of the imaging apparatus in order to guarantee an acquisition of image data of a desired or correct sub-area of a region of the body and a high quality of the acquired image data. The inventive method makes possible for the user an efficient and assisted choice of a diagnostically relevant sub-area of a region of the body for automated determination of an imaging protocol for acquisition of image data of the sub-area of the region of the body of the patient via an imaging apparatus.

In particular the proposed method can support the user patient-specifically in the selection of sub-areas of a region of the body. This can in particular be of advantage in an imaging examination of teeth, which can differ very greatly between patients and wherein, due to a high degree of symmetry between different tooth sections, confusions can easily occur.

Furthermore the inventive method can support the user in the determination of an imaging protocol as a function of different selected diagnostically relevant sub-areas of the regions of the body of the patient. For example by provision of an input option the diagnostically relevant sub-area of a region of the body can be made directly visible to a user and a standard parametrization of the imaging protocol for the diagnostically relevant sub-area can be defined automatically. The effort, in particular for the user, of creating an imaging protocol can advantageously be significantly reduced, and also the workflow to be carried out can be illustrated and simplified for a user. This enables the proposed method furthermore also to make it possible for a user of an imaging apparatus to determine, without specific prior knowledge, an imaging protocol for a diagnostically relevant sub-area. In particular the efficiency of a process of acquisition of the diagnostically relevant sub-area of a region of the body can be enhanced via provision of the input option. Errors during the determination of an imaging protocol and a subsequent acquisition of further image data of the diagnostically relevant sub-area of a region of the body can thus advantageously be avoided and further a high quality of the image data of the sub-area of the region of the body of the patient acquired via the imaging apparatus is guaranteed.

In a possible form of embodiment of the inventive method the acquisition of the information about the region of the body includes an acquisition of patient data and/or image data from previous examinations of the region of the body of the patient and/or an examination instruction.

The acquisition of the information about the region of the body of the patient can comprise a retrieval or receipt of data, in particular of patient data, image data from previous examinations of the region of the body of the patient and/or examination instructions, from an internal or external memory unit and/or a medical information system via an interface. A medical information system can for example represent a radiological information system (RIS) or a hospital information system (HIS). The imaging apparatus can preferably have a control unit and/or a processing unit, which is embodied to obtain the information about the region of the body of the patient as a function of patient data, image data from previous examinations of the region of the body of the patient and/or examination instructions via a suitable interface from the medical information system, from a Cloud and/or from a local memory unit.

The patient data can preferably comprise information about the patient and/or the region of the body of the patient and/or the sub-areas of the region of the body of the patient. The patient data can comprise a characteristic and/or condition of the patient, such as age, gender, weight and/or size of the patient, and/or a finding or a diagnosis, in particular information from findings or treatments that have been undertaken in the past, and/or any other medical or demographic information about the patient. The patient data can also be referred to as patient information or patient records. Patient data can in particular be available in the form of a digital record and/or collection of information on a memory medium and/or in a Cloud application. The information about the region of the body can be determined from patient data, preferably via an algorithm, in particular a text processing algorithm, and/or a trained function.

Image data from previous examinations of the region of the body of the patient can also be referred to as examination image data or localizer image data. Image data from previous examinations of the region of the body of the patient can comprise one or more image datasets consisting of one or more previous examinations. The image data can be available as raw data, preferably however as images and/or processed information that is derived from the raw data. Image data from previous examinations of the region of the body of the patient can preferably comprise image data of a localizer imaging. A localizer imaging can in particular be a time-efficient imaging examination, in which localizer image data of the region of the body of the patient is acquired. The localizer imaging can have a restricted or reduced quality and/or a spatial resolution of the acquired localizer image data when compared to a conventional imaging examination. Preferably the localizer imaging provides a spatial resolution, which is suitable for a detection and/or an identification of anatomical structures, in particular of sub-areas of a region of the body such as for example a tooth region, a dental arch, a jawbone or the like. The information about the region of the body can in particular be determined via an algorithm, in particular an image processing algorithm, and/or a trained function as a function of the image data from previous examinations of the region of the body of the patient, for example an imaging examination and/or localizer imaging of an examination already undertaken in the past.

An examination instruction can in particular comprise information about an imaging process or an imaging examination to be carried out. For example an examination instruction can comprise a result of an anamnesis and/or finding of a doctor in the form of a written examination report or a referral to another specialist area and/or doctor. An examination instruction can comprise a textual report, a written instruction, a doctor's marking (for example of a segmented area of a region of the body) and/or a doctor's or medical specialist's written note. In particular an examination instruction can comprise information about an examination (still to be performed) and/or the patient, which in particular is not to be taken from the patient data. Moreover examination instructions can be information that can be made available to a person that is not subject to medical confidentiality obligations. The information about the region of the body of the patient and/or possible relevant sub-areas can be determined in particular via an algorithm or a trained function, in particular for text recognition and/or processing, as a function of the examination instruction.

The algorithm mentioned here, which is embodied in particular for processing of the patient data, image data from previous examinations and examination instructions, can comprise a logic-based algorithm, a trained algorithm, a self-learning algorithm, an artificial neural network, a machine-learning algorithm and/or an image processing algorithm. The acquisition of the information about the region of the body of a patient can in particular comprise an algorithm and/or a trained function for acquisition of information from patient data, image data from previous examinations and/or an examination instruction.

Depending on the acquisition of information about a region of the body of a patient, there can in particular be a determination of sub-areas of the region of the body. In particular by way of a plurality of information about the region of the body of the patient there can be a determination of sub-areas. For example an input option with corresponding sub-areas determined can be created for selection by a user with the aid of the patient information. The patient-specific information of the region of the body of the patient can in particular be extracted by a trained function and/or an algorithm and on the basis of this there can be a determination of sub-areas of the region of the body.

An acquisition of the information about a region of the body via the acquisition of patient data, image data from previous examinations and/or examination instructions can advantageously make possible a simplified and/or improved acquisition of the information about a region of the body of a patient. In particular an automation of the acquisition of a region of the body of a patient, but also preferably the determination of the potentially diagnostically relevant sub-area is made possible by processing of the said information via an algorithm. Thus existing technical information about a patient can be used for individualization and adaption of the workflow of a determination of an imaging protocol for a user of an imaging apparatus.

In one possible form of embodiment of the inventive method the determination of the imaging protocol for acquisition of image data of the diagnostically relevant sub-area of the region of the body of the patient as a function of the diagnostically relevant sub-area acquired makes it possible to select an imaging protocol from a plurality of imaging protocols stored in a database.

For example a control unit and/or a processing unit of the imaging apparatus can be embodied to select and provide a specific imaging protocol from a plurality of imaging protocols stored in a library or a database as a function of the acquired diagnostically relevant sub-areas of the region of the body of the patient. Preferably the control unit and/or the processing unit can be embodied, as a function of the acquired information about the region of the body, to determine an imaging protocol for each of the sub-areas of the region of the body and store it in a database. It is also conceivable however for a manual selection of an imaging protocol to be made by a user from a plurality of imaging protocols provided by a memory unit and/or processing unit.

A database can in particular be a memory unit of the imaging apparatus, which is embodied to store one or more imaging protocols. For example a control unit and/or a processing unit of the imaging apparatus can comprise a memory unit. For each possible sub-area of a region of the body able to be imaged by an imaging examination a parametrized imaging protocol can be held or stored in the database. The stored imaging protocols can be comprehensively or only partly parameterized. In particular an imaging area of the diagnostically relevant sub-areas can be imaged and/or predetermined by a parameterized imaging protocol.

A selection of an imaging protocol from a plurality of imaging protocols stored in a database as a function of the diagnostically relevant sub-area of a region of the body can advantageously make possible the determination of an imaging protocol suitable for the diagnostically relevant sub-area of the region of the body. The parametrization and storage of imaging protocols in a database, after acquisition of an input of information about a sub-area of a region of the body by a user, enables the imaging protocol to be provided and/or adapted in a time-efficient manner. This can make possible a reduction of the effort for creation of an imaging protocol and/or a simplification of the workflow for determining an imaging protocol.

In one possible form of embodiment of the inventive method the determination of the imaging protocol for acquisition of image data of the diagnostically relevant sub-area of the region of the body of the patient as a function of the diagnostically relevant sub-areas acquired comprises a determination of an imaging parameter of the imaging protocol.

An imaging protocol comprises at least one imaging parameter. An imaging parameter can likewise be referred to as a parameter or imaging protocol parameter or protocol parameter. An imaging protocol enables the execution sequence and the characteristics of an imaging examination, in particular of imaging sequences of an imaging examination, to be defined. The imaging parameters can preferably make possible an activation of components of an imaging apparatus. Imaging parameters can comprise information about the spatial resolution, a contrast, a slice thickness, a dimension of an imaging volume, a relaxation time, an echo time and the like. An imaging parameter can in particular comprise any given image-relevant setting of the imaging apparatus, but also a parameter of a workflow of the imaging examination. In particular an imaging area of a diagnostically relevant sub-area can be defined by imaging parameters.

The determination of an imaging parameter of the imaging protocol can comprise selection of a parameter (and/or a group of parameters) from a database, adaptation of a parameter and/or the acquisition of an entry of a parameter. The selection of a parameter and/or of a group of parameters can be undertaken for example when a partly parameterized imaging protocol is present. Imaging parameters can preferably be selected from a database as a function of an acquired diagnostically relevant sub-area and added to the imaging protocol. For example an imaging parameter for a region of the body can have the same value for all sub-areas identifying the area of the body, so that the imaging parameters can just be stored once in the database and transferred into each imaging protocol of a sub-area. A parameter can preferably be selected from a database without user interaction.

The adaptation of a parameter can comprise a change to and/or removal of a parameter. For example an imaging parameter value of a completely parametrized imaging protocol can be changed as a function of the acquired diagnostically relevant sub-area. For example as a result of patient information a parameter of the imaging protocol can be adapted as a function of the acquired diagnostic sub-area.

The determination of an imaging parameter can comprise the acquisition of an input of a parameter and/or a change to a parameter by a user. A display unit, in particular a monitor, can be embodied to provide a completely or partly parameterized imaging protocol. The display unit can moreover comprise an input option for adaptation of a parameter of an imaging protocol. In particular only imaging parameters that need checking and/or adaptation by a user can be displayed by the display unit. For example more than one value can be stored in a database for an imaging parameter, so that a value can be selected by a user.

A determination of an imaging parameter of the imaging protocol can make possible an explicit adaptation of one of more parameters of an imaging protocol for a diagnostically relevant sub-area of the region of the body of the patient. By selecting an imaging protocol from an imaging protocol stored in a database, it is not possible for the individual characteristics of a patient, in particular of a diagnostically relevant sub-area of a region of the body of the patient, to be depicted adequately by the imaging protocol of the diagnostically relevant sub-area, for example. Advantageously, by adaptation of an imaging parameter, it is made possible for a user to adapt an imaging protocol in a time-efficient manner. This can mean a reduction in the effort for creation of an imaging protocol and/or a simplification of the workflow for determining an imaging protocol.

In one possible form of embodiment of the inventive method the acquisition of the diagnostically relevant sub-area as a function of the input option for acquisition of information about the sub-area of the region of the body comprises an acquisition of a plurality of diagnostically relevant sub-areas. The determination of the imaging protocol for acquisition of image data of the diagnostically relevant sub-area of the region of the body of the patient as a function of the diagnostically relevant sub-areas therefore comprises a determination of an imaging protocol for each of the acquired diagnostically relevant sub-areas. The provision of the imaging protocol for the acquisition of image data of the diagnostically relevant sub-area of the region of the body via the imaging apparatus comprises a provision of an imaging protocol for each of the acquired diagnostically relevant sub-areas.

In other words a selection of more than one diagnostically relevant sub-area is preferably possible for the user through the input option. The diagnostically relevant sub-areas here can in particular be spaced apart from one another, but sub-areas adjoining and/or neighboring to one another can also be selected. Spaced-apart diagnostically relevant sub-areas acquired can in particular be characterized in that one or more sub-areas are arranged between the acquired diagnostically relevant sub-areas.

Preferably the acquisition of a plurality of diagnostically relevant sub-areas can comprise a determination of the positioning and/or arrangement of the acquired diagnostically relevant sub-areas. The determination of the positioning and/or arrangement of the acquired diagnostically relevant sub-areas enables it to be determined whether the determination of the imaging protocol and provision of the imaging protocol should be performed for each of the diagnostically relevant sub-areas. In particular a prioritization or order of the imaging examinations and/or sequences can be undertaken from the positioning and/or arrangement of the acquired diagnostically relevant sub-areas. As an alternative, a prioritization or order of the imaging examinations and/or sequences can be entered manually by a user. For example the order in which the user has selected the diagnostically relevant sub-areas and the prioritization or order of the imaging examinations and/or sequences can be undertaken accordingly. Preferably there is a determination of the imaging protocol for each of the acquired diagnostically relevant sub-areas for specific spaced-apart positioning of the acquired diagnostically relevant sub-areas. The order of the image sequences due to the selected sub-areas can preferably be illustrated interactively to the user by the input option by a colored code and/or a graphical illustration.

The simultaneous creation of an imaging protocol for each of the diagnostically relevant sub-areas determined can make a simplified workflow possible for the user. This can make possible a time-efficient and cost-efficient determination of the imaging protocols with a simultaneous reduction of errors (for example through a duplicated determination of an imaging protocol for a sub-area). Especially advantageously this is possible for the presence of a complex or non-typically shaped region of the body of a patient, for example a jaw region, by which inexperienced users can typically be confused.

In one possible form of embodiment of the inventive method the acquisition of the diagnostically relevant sub-area as a function of the input option for acquisition of the information about a sub-area of the region of the body comprises an acquisition of a plurality of diagnostically relevant sub-areas. The method further has a determination of a common imaging area of the region of the body with the aid of a plurality of acquired diagnostically relevant sub-areas. The determination of the imaging protocol for acquisition of image data of the diagnostically relevant sub-areas of the region of the body of the patient is undertaken as a function of the common imaging area.

In other words a selection of more than one sub-area, for example of two sub-areas of the region of the body, by the input option is preferably possible for the user. The sub-areas here can in particular adjoin one another and/or be neighboring to one another, but a selection can also be made from sub-areas spaced apart from one another. The sub-areas can overlap and/or encompass one another.

Preferably the acquisition of a plurality of diagnostically relevant sub-areas can comprise a determination of the positioning and/or arrangement of the acquired diagnostically relevant sub-areas. The determination of the positioning and/or arrangement of the acquired diagnostically relevant sub-areas in particular enables it to be determined whether the determination of the imaging protocol and the provision of the imaging protocol can be undertaken via a common imaging protocol of the sub-areas. For a determined neighboring arrangement of the acquired sub-areas in particular, there can be the determination of a common imaging area of the region of the body. The determination of the common imaging area of the region of the body can in particular be undertaken as a function of the two or more acquired sub-areas of the regions of the body. If for example two of the acquired sub-areas of a region of the body overlap, in particular a common imaging area can be determined in order to avoid the repeated imaging of a sub-area of a region of the body of a patient.

The common imaging area can preferably comprise one or more diagnostically relevant sub-areas of the region of the body of the patient. In particular the common imaging area can define a volume within a patient receiving area of the imaging apparatus from which image data is acquired via an imaging examination. Preferably one or more diagnostically relevant sub-areas of the region of the body of the patient are positioned for an imaging examination in a so-called imaging volume of the imaging apparatus. A parameter of the common imaging area can for example define a spatial position, an alignment, a dimension, and/or a shape of the common imaging area. The common imaging area can in particular comprise a measurement volume, a field-of-view, a view window or a slice alignment. The common imaging area can also be referred to as the imaging area.

The input option can, in particular for a selection of a plurality of diagnostically relevant sub-areas, comprise a graphical representation of the common imaging area, in order to illustrate to the user the imaging volume of an imaging apparatus. The input option can comprise an output via the output unit of a user interface of information about a recommended or ideal selection of one or more sub-areas and/or about an ideal positioning of the common imaging area. For example a control unit and/or a processing unit of the imaging apparatus can be embodied to select and provide specific information about a desired or ideal setting of the common imaging area from a library or a database as a function of the region of the body of the patient. On the basis of this information the user can preferably be prompted to make an ideal selection of (diagnostically relevant) sub-areas.

The simplified determination of an imaging protocol for a common imaging area of the specific diagnostically relevant and preferably adjacently arranged sub-areas can make possible a reduction of imaging examinations to be carried out. Through the assisted determination of a common imaging area or of an imaging protocol for a common imaging area a time-efficient and cost-efficient workflow for determination of the imaging protocol with simultaneous reduction of manual inputs by a user is made possible. This is especially advantageous for the presence of a complex or non-typically shaped region of the body of a patient, for example a jaw region, for which a creation of an imaging protocol could typically not be able to be carried out by an inexperienced user.

In one possible form of embodiment the inventive method further comprises a provision of a further input option for acquisition of further information about the diagnostically relevant sub-area. Further included in the inventive method is an acquisition of an input as a function of the input option for acquisition of the further information about the diagnostically relevant sub-area. The establishment of the imaging protocol for acquisition of image data of the diagnostically relevant sub-area of the region of the body of the patient is additionally undertaken as a function of the information.

The further input option enables input of further information about a sub-area of the region of the body to be made possible for the user that is necessary and/or expedient for the determination of the imaging protocol for the sub-area of the region of the body. In particular the further input option can comprise a graphical representation of a sub-area of a region of the body and/or a text-based input option. In particular by way of a text-based input option it can be made possible for the user for example to enter keywords or brief information, which is recognized by an algorithm, in particular a trained function, and as a function of which the determination of the imaging protocol is additionally established.

The further input option can in particular be optional. The method can only comprise a further input option in application cases with in particular complex anatomical structure of the region of the body. In the determination of the imaging protocol for a sub-area of a region of the body of a patient in particular the case can arise in which more than one parameterization option for the imaging protocol exists or is stored in a database. In this case a selection of one of the options can be made possible for a user by the further input option. Preferably the method only comprises one further input option, it is however conceivable for any given number of optional further input options to be comprised by the method.

One further input option can simplify the workflow of the imaging protocol determination for a sub-area of a region of the body. In particular in application cases in which an automatic determination of the imaging protocol is not possible without further user input, the further input option can ensure that a time-efficient acquisition of the necessary information for imaging protocol determination is possible. Moreover, through a further input option, in particular a further specification option of the imaging protocol is made possible for an experienced user. A further input option can therefore increase the flexibility as regards the possible application cases, in particular as regards the regions of the body and/or sub-areas of regions of the body able to be imaged.

In one possible form of embodiment of the inventive method the further input option for acquisition of the further information about the diagnostically relevant sub-area comprises acquisition of information about an imaging parameter of the imaging protocol and/or a number of imaging examinations and/or an order of imaging examinations and/or a part area of the sub-area of the region of the body of the patient.

In the parameterization of an imaging protocol for a sub-area of the region of the body an imaging parameter of the imaging protocol can be unknown or not able to be parametrized in a unique way. There can be no information about the imaging parameter stored in a database for example. A number of items of information and/or options for an imaging parameter can however also be stored in a database for example. But also, because of a selection of a plurality of (diagnostically relevant) sub-areas, information about an imaging parameter can be unknown and/or a number of parameterization options can be available. The further input option can make possible the input and acquisition of further information with regard to an imaging parameter by a user. The further input option can in particular be designed in the form of a window that is only shown to a user when an imaging parameter is unknown or when a number of parameterization options for the imaging parameter have been established. The window can show the user a text input of the information of the imaging parameter and/or possible parameterization options and/or make it possible to select a parameterization option.

In particular with the selection of a plurality of diagnostically relevant sub-areas by a user, information about a number of imaging examinations can be required. The further input option can in particular be designed in the form of a window and/or a note, which is only displayed to the user when a plurality of sub-areas are selected. The further input option can also comprise a prompt before the acquisition of the information about one (or more) sub-areas of the region of the body. The prompt can for example represent a selection possibility as to whether a plurality of imaging examinations is permitted or desired by a user. For example the input option can comprise a threshold value or a maximum value of permitted imaging examinations. This makes it possible for a user for example to select between a time-efficient first option (by just one permitted imaging examination) or a second option with more detailed image recordings (by a specific number of imaging examinations).

In particular a selection of a plurality of diagnostically relevant sub-areas by a user can result in the determination of a plurality of imaging protocols for a plurality of imaging examinations. In this case the order of the imaging examinations can be able to be defined by a further input option. This can be of relevance since by an exploration of imaging examinations that have already happened during a series of imaging examinations, imaging examinations can be classified as no longer needed and can be aborted. A further load on the patient by an imaging examination no longer needed can thus be avoided. In other application cases there can be a temporally limited imaging examination with lower image quality, in order to make possible further findings or to plan the further treatments of the patient. A graphical, for example in the form of a timeline, or textual representation, for example in the form of a table, of the order of the imaging protocols can be comprised by the further input option. For example a graphical sorting and/or prioritization of the imaging protocols and/or the textual input of an order can be made possible.

The further input option, similarly to the first input option, can make it possible for the user to select a sub-area of a region of the body or a part area of a sub-area. For example the user can select a sub-area of a region of the body, which is subdivided however into further part areas by the first input option. The further input option can therefore make it possible for the user for example to select one or more part areas of the sub-area. Through the first input option for example the determination of the sub-areas of the upper and lower jawbone of a jaw region of a patient as diagnostically relevant sub-areas can have been undertaken by a user in each case and a second input option, for example for selection of a part area of the front of the bone area containing the incisor teeth, can be provided for the respective sub-area of the jaw region of the patient.

The acquisition of the said information by a further input option can make possible a further individualization or patient-specific adaptation of the imaging protocol. This can make possible both an advantageous imaging examination for the patient, in particular through a reduction in the examination load, and also a flexible series of imaging examinations for medical personnel. In particular the examination process of the patient and/or the workflow of the determination of an imaging protocol in general is able to be optimized by a further input option.

In one possible form of embodiment of the inventive method the further input option for acquisition of the further information about the diagnostically relevant sub-area comprises establishing a provisional imaging area. The provisional imaging area is adapted to the diagnostically relevant sub-area.

The establishing of a provisional imaging area can comprise a determination of the provisional imaging area, a provision of provisional imaging area and an acquisition of a correction of the provisional imaging area by a user. The correction of the provisional imaging area can in particular be undertaken by the further input option for acquisition of further information about the diagnostically relevant sub-area.

The determination of the provisional imaging area can comprise determining a dimension, of a spatial arrangement and/or a spatial alignment of the imaging area relative to the region of the body of the patient, in particular of acquired relevant sub-areas of a region of the body. The determination of the provisional imaging area can in particular be undertaken by an algorithm, in particular a trained function.

Preferably an, in particular graphical, representation of the provisional imaging area can be provided and/or shown by the provisional of the further input option. The representation of the provisional imaging area can for example comprise a symbol, a graphical object or a graphical representation of the provisional imaging area. The representation of the provisional imaging area can comprise a window or a polygon. In particular the representation of the provisional imaging area can comprise an overlaying of the, in particular graphical, representation of the region of the body and/or of the sub-areas of the region of the body.

Preferably a provisional imaging area, in particular with a plurality of acquired diagnostically relevant sub-areas; is proposed to a user and a correction and/or adaptation of the provisional imaging area is made possible by the further input option. The, in particular graphical, representation of the provisional imaging area can make it possible for a user, by shifting the provisional imaging area, to adapt the provisional imaging area. For example a polygon can be shifted in a graphical representation of a region of the body, so that an acquired sub-area is completely included and a further acquired sub-area is partly included in the provisional imaging area. However the orientation of a polygon comprising two acquired sub-areas can also be adapted in a graphical representation by a user, in order in particular to adapt the recording angle of an imaging examination. The output of the graphical representation of the provisional imaging area to a user and an adaptation of the provisional imaging area by a user are preferably made possible by an output unit and/or a user interface of an imaging apparatus.

Through the further input option an adaptation of the provisional imaging area can be made possible for a user. An imaging area adapted by the user can increase the quality and/or efficiency of an imaging examination carried out via the imaging protocol. With complex regions of the body and/or acquired sub-areas in particular, an interactive adaptation possibility of the provisional imaging area can clarify and/or simplify the method of determining an imaging protocol for a user.

In one possible form of embodiment of the inventive method the provision of the input option for acquisition of the information about the sub-area of the region of the body as a function of the information about the region of the body comprises a provision of an input option by a graphical representation of the region of the body and/or a graphical selection of the sub-areas of the region of the body and/or a text-based selection of the sub-areas of the region of the body.

A graphical representation of the region of the body can comprise pictographic, schematic and/or pictorial information about an area of the body and in particular pictographic, schematic and/or pictorial information about the sub-areas of the region of the body. The graphical representation can comprise information about the desired or ideal imaging parameters for an imaging protocol and/or help and/or (graphical) instructions for selection of a diagnostically relevant sub-area of the region of the body by the user.

A graphical selection of the sub-area of the region of the body enables a diagnostically relevant sub-area to be proposed to the user. Through a graphical selection of the sub-areas of the region of the body a selection of a sub-area, for example via an acquisition of a selection of a sub-area in a graphical representation of the region of the body, in particular by clicking on it, can be made possible for the user by a user interface. Preferably the graphical selection of the sub-area of the region of the body comprises a colored marking and/or outline of a sub-area of the region of the body.

Through the provision of a text-based and/or of a graphical selection of the sub-area and/or a graphical representation of a region of the body even inexperienced users can be informed about a standard parameterization of the imaging protocol as a function of the diagnostically relevant sub-areas of the region of the body. This enables costs for training of personnel, but also errors in the determination of the imaging protocol, to be reduced in an advantageous way.

In one possible form of embodiment of the inventive method the graphical representation of the region of the body and/or the graphical selection of the sub-area of the region of the body the input option comprises a two-dimensional representation of the region of the body and/or of the sub-areas of the region of the body. In particular the two-dimensional representation can represent a sectional representation.

The two-dimensional representation of the region of the body can comprise a simplified and/or schematized representation of the region of the body. The two-dimensional representation can comprise a simplified and/or schematized sectional representation of the region of the body. In particular the two-dimensional representation can comprise two sectional representations of the region of the body of the patient, which are preferably aligned along different reference planes, preferably aligned orthogonal to one another. The reference planes can comprise the sagittal plane, the frontal plane and the transversal plane of the patient. For example the two-dimensional representation of the region of the body can comprise a representation of the lower and upper jawbone area of a patient arranged above one another with the transversal plane of the patient as reference plane.

A provision of a two-dimensional representation of the region of the body and/or of the sub-areas of the region of the body can allow an illustration of the region of the body and/or of the sub-areas in at least two reference planes. This makes it possible for a user of the imaging apparatus in an advantageous way to recognize the arrangement of the sub-areas of the region of the body taking into account an individual nature of the region of the body of the patient and to make a selection of the diagnostically relevant sub-areas accordingly. In particular a schematic two-dimensional representation of the region of the body and/or of the sub-areas of the region of the body can lead to a simplified recognition of a diagnostically relevant sub-area by a user, since despite taking into account the individual nature of the patient, there only has to be the recognition of a schematic representation similar as a rule for most patients.

In one possible form of embodiment of the method the region of the body of the patient comprises a jaw region and/or a tooth region.

For example the jaw region and/or the tooth region have a set of teeth, a section of a jawbone, a section of a set of teeth, one or more dental arches, a section of a dental arch, a gingiva, section of a gingiva and/or one or more teeth of the patient.

A tooth region of a patient has two essentially same-shaped dental arches, which in turn have section on the left half of the body of the patient and the right half of the body of the patient. Moreover rows of front teeth of the dental arch are arranged at an angle to rows of back teeth of the dental arch, which is why numerous sections of the tooth region must be distinguished in the imaging examination of the tooth region. The sub-areas of a jaw region and/or a tooth region can in particular appear to be optically similar for an inexperienced user, whereby a risk of confusing a right side with a left side of a dental arch or of a section of a lower tooth with a section of an upper dental arch is increased.

The provision of an input option for acquisition of information about a sub-area of the region of the body as a function of the Information about the region of the body enables a direct relationship to a sub-area of the region of the body relevant for the imaging examination to be provided. In an advantageous way this allows the risk of selecting an incorrect sub-area of the region of the body by a user of the imaging apparatus, even with complex anatomical structures such as a jaw region and/or a tooth region with different sections, to be reduced or avoided.

In a preferred form of embodiment the acquisition of image data of a diagnostically relevant sub-area comprises one or more magnetic resonance examinations of a jaw region or of a tooth region of a patient. It is conceivable for an imaging sequence of at least one magnetic resonance examination to have a very short echo time in order to compensate for a very short T2 relaxation time of spins of a dentine or of a dental enamel of a tooth and to represent these areas with high signal intensity in acquired image data. Very short echo times can be less than 150 μs or less than 70 μs. Possible imaging sequences for example represent FLASH (“fast low-angle shot”) or UTE (“ultra-short echo time”) sequences. It is however likewise conceivable for imaging sequences with a longer echo time, such as for example a TSE (“turbo spin echo”) sequence, to be used. With such sequences an acquisition of the magnetic resonance signal of the tooth enamel or of the dentine are avoided. In image data of such imaging sequences the teeth are able to be differentiated by a lack of signal intensity by comparison with a surrounding tissue.

In a preferred form of embodiment of the inventive method the acquisition of the information about the region of the body of the patient comprises an acquisition of a section of a tooth region, in particular of a section of one or more dental arches of the patient.

In a preferred form of embodiment of the inventive method the acquisition of image data of the region of the body is undertaken via an antenna element or a plurality of antenna elements of a magnetic resonance apparatus. The antenna elements can be positioned for this for example on a jaw region and/or in a mouth cavity of the patient. The antenna element or the plurality of antenna elements can in particular be designed to receive magnetic resonance signals of the jaw region and to transfer these to a receive unit of the magnetic resonance apparatus.

The inventive imaging apparatus is embodied for acquisition of image data of a diagnostically relevant sub-area of a region of the body of a patient. The imaging apparatus comprises a control unit, an output unit and a user interface. The control unit is embodied to carry out a method in accordance with one of the forms of embodiment described above via the imaging apparatus. The output unit is embodied to provide a user of the imaging apparatus with the input option for acquisition of the information about the sub-area of the region of the body. The user interface is embodied to establish and to provide the imaging protocol as a function of the diagnostically relevant sub-areas.

In a preferred form of embodiment the inventive imaging apparatus is embodied as a magnetic resonance apparatus. Preferably the magnetic resonance apparatus can be embodied to acquire magnetic resonance data, in particular magnetic resonance images, of a patient positioned in a patient receiving area of the magnetic resonance apparatus. The magnetic resonance data or magnetic resonance image data can comprise image data in accordance with a form of embodiment described above.

The magnetic resonance apparatus is embodied to carry out one or more imaging examinations of one and/or more sub-areas of the region of the body of the patient in accordance with a form of embodiment of the proposed method described above. The use of a magnetic resonance apparatus enables a load on the patient with ionizing radiation to be avoided in an advantageous way by comparison with x-ray devices or computed tomography devices.

The control unit can preferably be embodied to coordinate a method in accordance with one of the forms of embodiment described above and to carry it out via the imaging apparatus. The control unit can preferably be integrated into the imaging apparatus or be embodied as a self-contained component.

The output unit is preferably embodied to display an input option to a user of the imaging apparatus, in particular through a monitor. The output unit can preferably be embodied to provide a user of the imaging apparatus with a further input option and in particular to display it to them through a monitor. The user interface can be embodied to make possible an input or a selection of a diagnostically relevant sub-area for the user. The user interface can be embodied to make the adaptation of a (common) imaging area possible for a user depending on a further input option.

The output unit and the user interface can preferably be integrated into one component. The output unit and the user interface can however also be embodied as self-contained components. In a preferred form of embodiment the output unit and the user interface form a graphical user interface, which is embodied to make it possible for the user of the imaging apparatus to have an input option for provision of acquisition of information about a sub-area of a region of the body and to select a diagnostically relevant sub-areas.

Preferably the control unit has a signal connection to the user interface and/or to the output unit of the imaging apparatus. In particular the control unit can be embodied to activate the output unit to output the input option to the user of the imaging apparatus. The control unit can furthermore be embodied to acquire diagnostically relevant sub-areas selected by the user via the user interface.

The proposed imaging apparatus has the advantages of a proposed method in accordance with one of the forms of embodiment described above.

The components of the proposed imaging apparatus can advantageously be matched to one another, so that a time-efficient and robust execution of a proposed method is made possible. In particular the imaging apparatus can be embodied to coordinate and carry out a method sequence autonomously. Thus it is made possible for the user of the imaging apparatus to select an imaging area for a further imaging examination by selection of a sub-area of the region of the body of the patient without the user needing any expert knowledge or expensive training being necessary.

The inventive non-transitory computer program product is able to be loaded directly into a memory unit of a processing unit of a proposed imaging apparatus. The computer program product has program code for carrying out a method in accordance with one of the forms of embodiment described above when the computer program product is executed in the processing unit of the imaging apparatus.

The proposed computer program product enables the proposed method to be carried out quickly, identically repeatably and robustly. The computer program product is configured so that, via the processing unit, it can carry out the proposed method steps. Preferably the processing unit has the necessary prerequisites, such as for example a main memory, a graphics card or a logic unit, so that the respective method steps can be carried out efficiently. The computer program product is for example stored on a computer-readable medium or in a network, a server or a Cloud, from where it can be loaded into a processor of the processing unit. The processing unit in this case can be embodied as a self-contained system component or as a part of the imaging apparatus. Furthermore control information of the computer program product can be stored on an electronically-readable data medium. The control information of the electronically-readable data medium can be embodied in such a way that, when the data medium is used in the processing unit of the imaging apparatus, it carries out a proposed method. Examples of electronically-readable data media are a DVD, a magnetic tape, a USB sick or any other data medium on which electronically-readable control information, in particular software, is stored. When this control information is read from the data medium and transmitted to a control unit and/or the processing unit of the imaging apparatus, all proposed forms of embodiment of the proposed method described can be carried out.

### BRIEF DESCRIPTION OF THE DRAWINGS

Further advantages and details of the present invention emerge from the exemplary embodiments described below and also with the aid of the drawings. In the figures:

FIG. 1 shows a schematic representation of a form of embodiment of a proposed imaging apparatus,

FIG. 2 shows a flow diagram of a form of embodiment of a proposed method,

FIG. 3 shows a flow diagram of a form of embodiment of a proposed method with optional method steps,

FIG. 4 shows a schematic representation of an input option for acquisition of information about a sub-area in a form of embodiment of a proposed method with acquisition of a diagnostically relevant sub-area,

FIG. 5 shows a schematic representation of an input option for acquisition of information about a sub-area in a form of embodiment of a proposed method with acquisition of two spaced-apart diagnostically relevant sub-areas,

FIG. 6 shows a schematic representation of an input option for acquisition of information about a sub-area in a form of embodiment of a proposed method with acquisition of two neighboring diagnostically relevant sub-areas with a common imaging area,

FIG. 7 shows a schematic representation of an input option for acquisition of information about a sub-area in a form of embodiment of a proposed method with acquisition of two neighboring diagnostically relevant sub-areas each with imaging areas,

FIG. 8 shows a schematic text-based representation of an input option for acquisition of information about a sub-area in a form of embodiment of a proposed method with a further input option.

### DETAILED DESCRIPTION

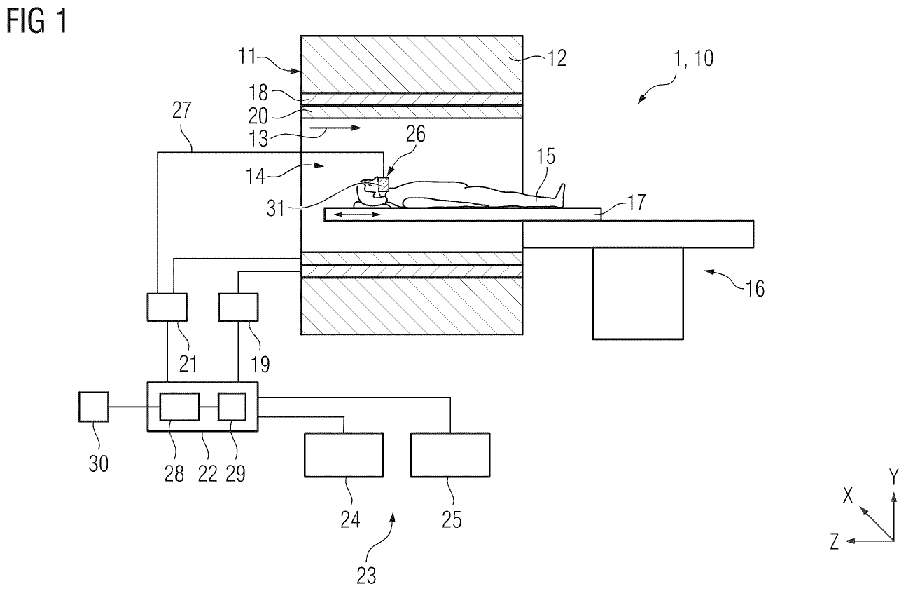

Shown in FIG. 1 is a form of embodiment of a proposed imaging apparatus 1. The imaging apparatus 1 is embodied here as a magnetic resonance apparatus 10. The magnetic resonance apparatus 10 comprises a magnet unit 11, which for example has a permanent magnet, an electromagnet or a superconducting main magnet 12 for generating a strong and in particular homogeneous main magnetic field 13 (B0 magnetic field). Moreover the magnetic resonance apparatus 10 comprises a patient receiving area 14 for receiving a patient 15. The patient receiving area 14 in this exemplary embodiment is embodied in a cylindrical shape and is surrounded in a circumferential direction by the magnet unit 11. Basically however embodiments of the patient receiving area 14 that differ from this example are conceivable.

The patient 15 can be positioned via a patient support apparatus 16 of the magnetic resonance apparatus 10 in the patient receiving area 14. For this, the patient support apparatus 16 has a patient table 17 able to be moved within the patient receiving area 14.

The magnetic resonance apparatus 10 furthermore has a gradient coil 18 for creation of magnetic gradient fields, which are used for a spatial encoding during a magnetic resonance measurement. The gradient coil 18 is activated via a gradient control unit 19 of the magnetic resonance apparatus 10.

The magnetic resonance apparatus 10 can furthermore comprise a radio-frequency antenna, which in the present exemplary embodiment is embodied as a body coil 20 permanently integrated into the magnetic resonance apparatus 10. The body coil 20 is designed to excite nuclear spins, which are to be found in the main magnetic field 12 created by the main magnet 13. The body coil 20 is controlled by a radio-frequency unit 21 of the magnetic resonance apparatus 10 and emits radio-frequency excitation pulses into an imaging region, which is essentially formed by the patient receiving area 14 of the magnetic resonance apparatus 10. The body coil 20 can be embodied as a receive unit of the magnetic resonance apparatus 10, which is embodied to receive magnetic resonance signals from the patient receiving area 14.

For control of the main magnet 12, the gradient control unit 19 and the radio-frequency unit 21, the magnetic resonance apparatus 10 has a control unit 22. The control unit 22 is embodied to control the execution of an imaging sequence of the imaging examination, such as for example a GRE (gradient echo) sequence, a TSE (turbo spin echo) sequence or a UTE (ultra-short echo time) sequence. Moreover the control unit 22 comprises a processing unit 28 for an evaluation of magnetic resonance signals that are acquired during a magnetic resonance examination.

Furthermore the magnetic resonance apparatus 10 comprises a user interface 23, which has a signal connection to the control unit 22. Control information, such as for example imaging parameters of the magnetic resonance examination, but also reconstructed image data of a region of the body 31 of the patient 15, as well as help for adaptation of an imaging parameter, can be displayed on a output unit 24 (also referred to as a display unit) of the user interface 23 for a user. For this, the output unit 24 can for example comprise one or more monitors. The output unit 24 can in particular be designed to provide a graphical user interface with schematic image data of the region of the body 31 of the patient and an input option for selection of a diagnostically relevant sub-area. Preferably the user interface 23 has an input unit 25, which makes possible for the user in particular a selection of a diagnostically relevant area and/or an adaptation of imaging parameters. The input unit 25 can further be embodied to make possible for the user an adaptation of a dimension, an alignment and/or a position of a graphical object that represents a common imaging area of a number of sub-areas of a region of the body, as a function of acquired sub-areas (32a, 32b) of the region of the body 31 of the patient 15 and a further input option for adaptation to the common imaging area.

The processing unit 28 in the present example is connected via a signal connection to a memory unit 29 of the magnetic resonance apparatus 10. Optionally the processing unit 28 can also be connected via a signal connection to a Cloud 30. The processing unit 28 can be embodied to store data such as patient information, image data, localizer image data, magnetic resonance images, x-ray images or the like, on the memory unit 29 and/or the Cloud 30 and/or to retrieve corresponding data from the memory unit 29 and/or the Cloud 30 via a suitable interface (not shown). It is conceivable for the processing unit 28 to be embodied to obtain patient information of the patient 15 from the Cloud 30 and/or the memory unit 29. It is likewise conceivable for the processing unit 28 to be embodied to obtain information about the region of the body 31 of the patient 15 as a function of patient information, in particular a name and/or another identification, from the Cloud 30 and/or the memory unit 29.

In a preferred form of embodiment the magnetic resonance apparatus 10 has a dental coil 26, which is positioned in an application-dictated position on the jaw region 31 and/or in in a mouth cavity of the patient 15. The dental coil 26 can have an antenna element (not shown), which is embodied to acquire magnetic resonance signals of the jaw region and/or the tooth region of the patient 15 and transfer them to the processing unit 28 and/or the control unit 22.

The dental coil 26 in the present case has an electrical connecting lead 27, which provides a signal connection to the radio-frequency unit 21. In a preferred form of embodiment the dental coil 26 is embodied to excite nuclear spins in the jaw region 31 of the patient 15. For this, the dental coil 26 can be activated by the radio-frequency unit 21. In one example the dental coil 26 is embodied as a mask, which is positioned in an application-related position on a skin surface of the jaw region 31 of the patient 15. It is likewise conceivable however for the dental coil 26 to be connected mechanically to a bite element, which is positioned in an application-dictated position on a dental arch, in particular along an occlusion plane, of the patient 15.

The proposed magnetic resonance apparatus 10 can naturally comprise further components that magnetic resonance apparatuses usually have. It is likewise conceivable for the magnetic resonance apparatus 10, instead of the cylindrical structure, to have a C-shaped, a triangular or an asymmetrical structure of the components creating the magnetic field. The magnetic resonance apparatus 10 can in particular be embodied to carry out a magnetic resonance examination of a standing or seated patient 15.

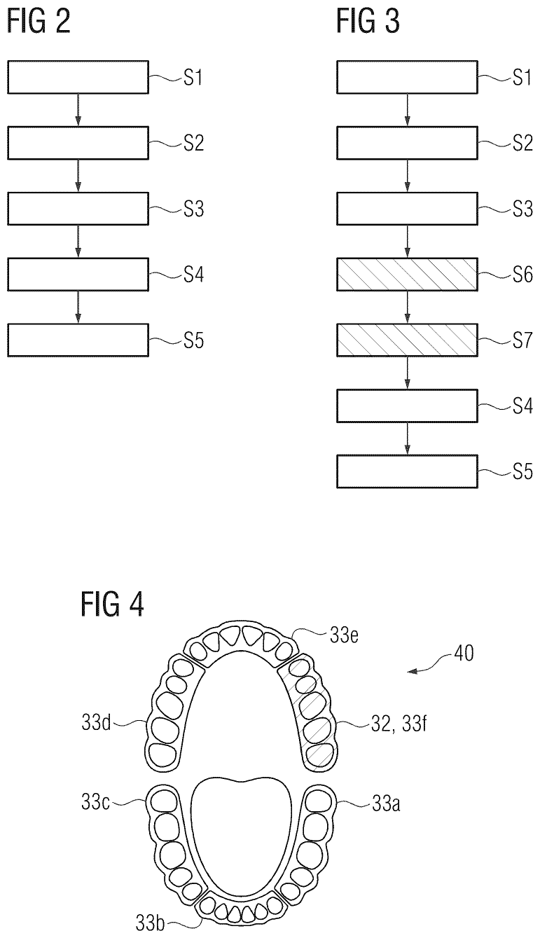

Shown in FIG. 2 is a flow diagram of a form of embodiment of the proposed method for acquisition of image data of a diagnostically relevant sub-area of a region of the body 31 of a patient. Shown in FIG. 3 is a flow diagram of a further form of embodiment of the proposed method for acquisition of image data of a diagnostically relevant sub-area of a region of the body of a patient 15 with optional method steps. The execution sequence of the exemplary forms of embodiment of the method is explained below with the aid of a magnetic resonance apparatus 10. Naturally the inventive method can also be carried out via another imaging apparatus in accordance with the form of embodiment described above.

In step S1 information about the region of the body 31 of the patient is acquired. It is conceivable for the information about the region of the body of the patient to be input by a user of the magnetic resonance apparatus 10 via the user interface 23. For this, the user interface 23 can comprise any given input unit 25, which makes possible an interaction of the user with a graphical user interface. Preferably the input unit 25 has a keyboard, a mouse and/or a touchscreen. The information entered by the user about the region of the body of the patient can subsequently be acquired and processed via the control unit 22 and/or the processing unit 28 of the magnetic resonance apparatus 10. As an alternative the information about the region of the body of the patient can be preconfigured when the magnetic resonance apparatus 10 is only employed to record one region of the body. The information about the region of the body of the patient can likewise be acquired or requested, depending on patient information, from a radiological information system, a hospital information system, a network memory unit, a local memory unit 29 and/or a Cloud 30.

In a step S2 an input option is provided, through which information about a sub-area of the region of the body can be acquired. The provision of the input option is undertaken here as a function of the information about the region of the body. The region of the body comprises a number of sub-areas that are able to be selected through the input option. Preferably the input option for acquisition of information about the sub-area, in particular for a selection of a diagnostically relevant sub-area, is provided to the user of the magnetic resonance apparatus 10 via the output unit 25. As an alternative a parameter of a sub-area or also other information about a sub-area can be acquired. The acquisition of an adaptation of a parameter of a sub-area and/or other information can however in particular be undertaken via a further input option (see S6 and S7). The provision of the input option can in particular comprise a display of a schematic graphical depiction of the region of the body on a monitor of the user interface 23.

Preferably the graphical (or text-based) representation of the region of the body of the patient and the input option for acquisition of information about a sub-area represent a part of an output 40 (see FIGS. 3 to 7) of the user interface 23, which make it possible for the user to determine an imaging protocol of a magnetic resonance examination, in particular through determination of a diagnostically relevant sub-area and/or for example of a parameter of a common imaging area of a number of sub-areas for a imaging examination.

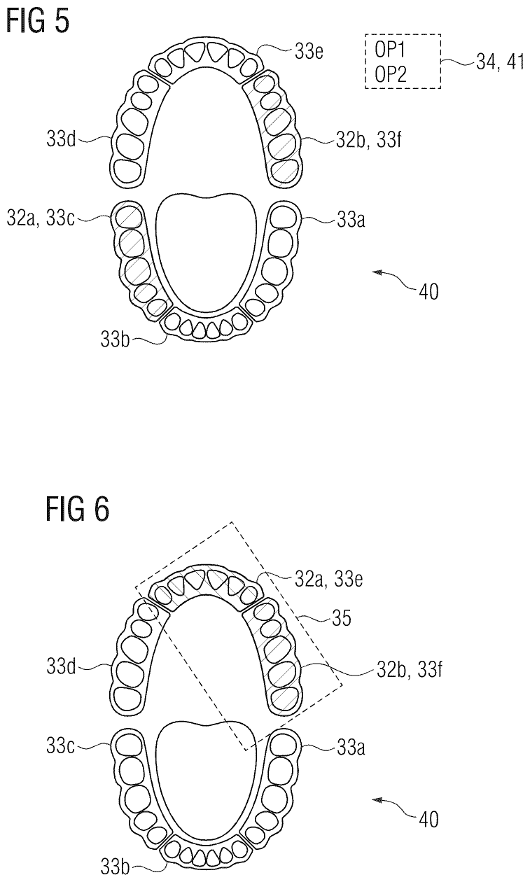

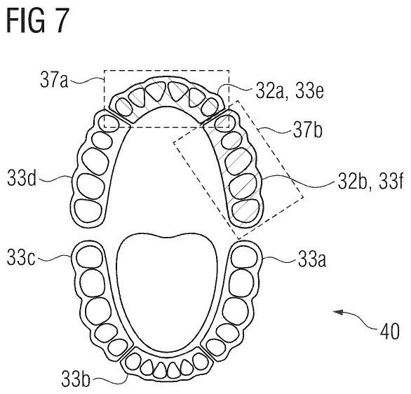

An input option for acquisition of information about a sub-area can for example comprise a text-based input mask and/or a text-based input window (see FIG. 7). Preferably the input option, which can likewise make possible an adaptation of a parameter of a (common) imaging area (see FIG. 6), can be a graphical representation of the region of the body or a graphical object (see FIGS. 4 to 6), which can make it possible for the user to select sub-areas.

In a further step S3 a diagnostically relevant sub-area of a region of the body is acquired. Preferably at least one sub-area of the region of the body selected by the user via the user interface 23 is acquired by the control unit 22 and/or the processing unit 28. The acquisition of information about a sub-area, in particular of a diagnostically relevant sub-area, can comprise an acquisition of an input via a mouse, a keyboard and/or a touch input of a touchscreen. The acquisition of the diagnostically relevant sub-area can in particular comprise a storage of the diagnostically relevant sub-area on the memory unit 29, a Cloud memory 30, a network memory or the like.

In an optional step S6 there can be the provision of a further input option for acquisition of further information about a diagnostically relevant sub-area of the region of the body. The further information about the diagnostically relevant sub-area can in particular comprise a parameter of a sub-area or of an imaging area and/or for example the selection of a part area of the sub-area of a region of the body of the patient. For example the control unit 22 and/or the processing unit 28 can be embodied, as a function of the sub-areas selected by the user to establish a selected provisional imaging area and/or a graphical object, which represents a common imaging area of a number of sub-areas. For example the user can adapt a dimension, an alignment and/or a position of the representation of the (common) imaging area in order to adapt the parameter of the (common) imaging area or of an imaging protocol of a sub-area. Preferably the control unit 22 and/or the processing unit 28 of the magnetic resonance apparatus 10 are embodied to derive or to establish the adaptation of the parameter with the aid of the representation of the (common) imaging area changed by the user.

Preferably the provisional imaging area can be established automatically via the control unit 22 and/or the processing unit 28 of the magnetic resonance device 10 as a function of the acquired diagnostically relevant sub-areas of the region of the body 31 of the patient 15. The control unit 22 and/or the processing unit 28 can include an algorithm, which is embodied, for one or more diagnostically relevant sub-areas of the region of the body 31 of the patient 15, to establish and/or provide a provisional and/or common imaging area.

In an optional step S7 an input can be acquired as a function of the further input option for acquisition of the further information about the diagnostically relevant sub-area. Preferably at least one adapted imaging parameter of the imaging protocol input by the user via the user interface 23 can be acquired by the control unit 22 and/or the processing unit 28. As an alternative the further information about the diagnostically relevant sub-area can comprise a number of imaging examinations, an order of imaging examinations or an acquired part area of a sub-area of a region of the body. The acquisition of further information about the diagnostically relevant sub-areas can comprise an acquisition of an input via a mouse, a keyboard and/or a touch input of a touchscreen. The acquisition of the further information can in particular comprise a storage of the further information on the memory unit 29, in a Cloud memory 30, a network memory or the like.

In a step S4 an imaging protocol for acquisition of image data of the diagnostically relevant sub-area of the region of the body of the patient is established. The establishing of the imaging protocol is undertaken for the acquired diagnostically relevant sub-area. The establishing of the imaging protocol for an imaging examination can in particular be undertaken automatically on the basis of the acquired diagnostically relevant sub-areas. Preferably the establishing of the imaging protocol for the imaging examination is carried out automatically via the control unit 22 and/or the processing unit 28. The processing unit 28 can include an algorithm, which is embodied to establish the imaging protocol for the imaging examination as a function of the acquired diagnostically relevant sub-areas. For example the algorithm can further be embodied to establish a change made by the user of a dimension, a position and/or an alignment of the provisional (or common) imaging area and to translate it into an imaging protocol for the imaging examination. The control unit 22 and/or the processing unit 28 can however likewise be embodied to establish the imaging protocol with the aid of numerical data and/or a text-based input of the user.

In a step S5 the established imaging protocol for acquisition of image data of the diagnostically relevant sub-area (32) of the region of the body (31) is provided via the imaging apparatus (1). The user interface 23; 25 and/or the processing unit 28 can preferably be embodied to provide the imaging protocol to the control unit 22. As an alternative the control unit 22 can also be embodied for provision of the imaging protocol. After the provision of the imaging protocol to the control unit 22, the control unit 22 can in particular control the execution of an imaging examination via the magnetic resonance apparatus 10 with the aid of the imaging protocol established.

For carrying out the imaging examination the patient 15, as shown in in FIG. 1, can be positioned in accordance with the application in the patient receiving area 14 of the magnetic resonance apparatus 10. The imaging examination can for example be a magnetic resonance examination of a sub-area of a tooth region, for example the incisor region, of the patient 15. Preferably a resolution via the image data of the diagnostically relevant sub-area acquired by the imaging examination, for example the incisor region, is higher than a resolution of the image data from the region of the body, for example the tooth region. Likewise the diagnostically relevant sub-area of the region of the body 31 of the patient 15 is better centered and/or focused on the image data than in the image data of an imaging examination of a region of the body 31 of the patient 15.

Shown in FIG. 4 is a schematic representation of an input option 40 for acquisition of information about a sub-area 33a, 33b, 33c, 33d, 33e, 33f in a form of embodiment of the proposed method with acquisition of a diagnostically relevant sub-area 32, 33f. The exemplary representation in FIG. 4 can in particular represent an output 40 of the output unit 25 and/or of the user interface 23 for a user of the imaging apparatus 1.

The provision of the input option 40 for acquisition of information about a diagnostically relevant sub-area 32, 33f in accordance with steps S2 here in particular comprises a graphical representation of the region of the body and a graphical selection of the sub-areas 33a-33f of the region of the body of the patient for selection of a diagnostically relevant sub-area 32, 33f of the region of the body. The diagnostically relevant sub-areas 32, 33f can be proposed automatically to the user as a function of the information about the region of the body 31, patient information, image data from previous examinations and/or an examination instruction and an ideal imaging area for a specific (diagnostically relevant) sub-area of the region of the body. In particular the graphical selection of a sub-area 33a-33f of the region of the body and/or a proposed sub-area of the region of the body can or will be marked in color in the input option. For example the control unit 22 and/or the processing unit 28 can be embodied to select or to establish the diagnostically relevant sub-areas 32, 33f as a function of the information about the region of the body 31 of the patient 15, patient information, image data from previous examinations and/or an examination instruction. The input option 40 for acquisition of information about a diagnostically relevant sub-area 32, 33f can, other than in the way shown, in particular comprise a graphical instruction for selection of one or more sub-areas 32, 33f of the region of the body.

Shown in FIG. 5 is a schematic representation of an input option 40 for acquisition of information about a sub-area 33a-33f in a further form of embodiment of the proposed method with acquisition of a diagnostically relevant sub-area 32, 33f. The exemplary representation in FIG. 4 can in particular represent an output 40 of the output unit 25 and/or of the user interface 23 for a user of the imaging apparatus 1.

By contrast with the form of embodiment shown in FIG. 4, the form of embodiment shows a further input option 41 which can be displayed in a text field or window 34. Moreover the form of embodiment shown in FIG. 5 illustrates the selection of more than one diagnostically relevant sub-area 32a, 33f, 32b, 33c via the input option 40. The diagnostically relevant sub-areas 32a, 33f, 32b, 33c determined by a user are arranged in this example spaced apart from one another, so that preferably an imaging protocol in accordance with a step S3 of a form of embodiment of the method proposed above is established in each case.

The input option 41 can in particular, for adaptation of a parameter of the imaging protocol for one of the acquired diagnostically relevant sub-areas 32a, 33f, 32b, 33c, comprise a text field 34 for input of parameter values. For example the parameter OP1 can define a dimension and the parameter OP2 an alignment of an imaging area of one of the diagnostically relevant sub-areas 32a, 33f, 32b, 33c. As well as this an input of further parameters, which are relevant for the respective imaging apparatus 1 are possible. It is furthermore conceivable for the input option 41 for adaptation of a parameter as shown in FIG. 6 to comprise a graphical object, which makes a graphical adaptation of a (common) imaging area 35 possible for the user. Preferably the parameters OP1, OP2 are updated depending on a manipulation of the graphical object by the user. As an alternative it is conceivable for the further input option 41 to provide a selection of, for example, two options OP1, OP2 for a user. In particular on the basis of the selection of diagnostically relevant sub-areas 32a, 33f, 32b, 33c via the input option 40 a further user input can be necessary. For example, as shown, spaced-apart diagnostically relevant sub-areas 32a, 33f, 32b, 33c can be determined by the user and determined through the further input option 41 and it can be shown interactively in a window 34 determined by the user as a result of the selection whether there should be a common imaging of the diagnostically relevant sub-areas 32a, 33f, 32b, 33c via a common imaging area according to a first option OP1 or the provision of two imaging protocols for two imaging examinations for each of the acquired diagnostically relevant sub-areas 32a, 33f, 32b, 33c according to a second option OP2 1. The second option is illustrated in FIG. 7.

Shown in FIG. 6 is a schematic representation of an input option 40 for acquisition of information about a sub-area 33a-33f in a further form of embodiment of the proposed method with acquisition of a diagnostically relevant sub-area 32, 33f. The exemplary representation in FIG. 4 can in particular represent an output 40 of the output unit 25 and/or of the user interface 23 for a user of the imaging apparatus 1.

By contrast with the forms of embodiment shown in FIG. 4 and FIG. 5, the form of embodiment of FIG. 6 comprises a further input option 41, which is embodied by the graphical representation of a common imaging area 35. The form of embodiment shown in FIG. 6 also illustrates the selection of more than one diagnostically relevant sub-area 32a, 33e, 32b, 33f via the input option 40. The diagnostically relevant sub-areas 32a, 33e, 32b, 33f determined by a user, in this example, by contrast with the depiction in FIG. 5, are arranged adjacent to one another, so that preferably an imaging protocol is established as a function of a common imaging area in accordance with a step S3 of a form of embodiment of the method proposed above.

The region of the body 31 can be represented as in FIG. 6 by two schematic sectional views of the region of the body 31 of the patient 15, but as an alternative can also be depicted by a three-dimensional model view. The input option 41 here is embodied as a graphical object, which is able to be manipulated by the user. Preferably a dimension, an alignment and/or a position of the graphical object updates the input option 41 when the user has undertaken an adaptation of the common imaging area 35.

A provision of an overview view, comparable with the depiction of the region of the body 31 in the FIGS. 4 to 6, is also conceivable for example for the step S1 as an input option for the user, in order to image different areas of the body of a patient 15 and to acquire the information about the region of the body 31 of the patient 15.

In one form of embodiment of the proposed method the provision of help, for adaptation of the common imaging area as a function of the acquired diagnostically relevant sub-areas of the region of the body 31 for example is included via an output of a text-based instruction. In the example shown in FIG. 6 the help can for example comprise the text-based instruction in the form of two instructions not shown, with said instructions comprising the execution sequence of the adaptation of the common imaging area 35. For example a first instruction instructs the user to position the common imaging area 35 in an advantageous alignment for an imaging examination, in particular at a specific angle. A second instruction can for example instruct the user to align the location and/or dimensioning of the common imaging area 35 so that all selected (marked) diagnostically relevant sub-areas are included in the common imaging area 35.

Shown in FIG. 7 is a schematic representation of an input option 40 for acquisition of information about a sub-area 33a-33f in a further form of embodiment of the proposed method with acquisition of a diagnostically relevant sub-area 32. The identification of two sub-areas 33e, 33f as diagnostically relevant sub-areas 32a, 32b by a user via the input option 40 is explained. For each of the diagnostically relevant sub-areas there can be an automatic adaptation of the imaging area to the anatomy of the sub-area.

In FIGS. 3 to 7 the region of the body 31 of the patient 15 is a jaw region and/or a tooth region of the patient 15. By way of example and not shown, one form of embodiment of the proposed method can however also relate to another region of the body, in particular to a spinal cord of the patient 15. Similarly to the forms of embodiment described above, the area of the spinal cord of the patient 15 can be depicted by a graphical representation of an input option 40 and make possible for the user a selection of a sub-area of the spinal cord, i.e. for example of the lower spinal cord area with three vertebrae included.

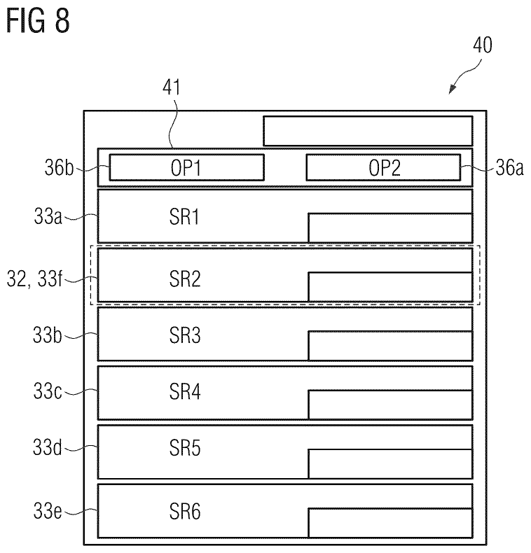

The input option 40 shown in FIGS. 4, 5, 6 and 7, or the graphical representation of the region of the body 31 of the patient 15 here can be a two-dimensional representation, in particular a sectional view, of the tooth region of the patient 15. The reference plane of the sectional view of the region of the body 31 of the patient 15, in the example shown, is the transversal plane of the patient 15. An alternative embodiment of the input option 40 as text-based selection of the diagnostically relevant sub-areas 32, 33f of the region of the body 31 is shown in FIG. 8. The input option in this form of embodiment comprises a list representation of the sub-areas 33a-33f of the region of the body. A selection of the diagnostically relevant sub-area 32, 33f is possible for a user for example through selection of a list element. The selected list element can preferably be made clear to the user by a surround or by a colored marking. The further input option 41 can also be embodied as a text-based selection. For example, it is possible for a user to select two selection options OP1, OP2 as text fields 36a, 36b.

Naturally the inventive method can be used for determining an imaging protocol for acquisition of image data of any other given regions of the body. In particular the inventive method is used for determining an imaging protocol for acquisition of image data from regions of the body with a plurality of differently aligned sections or sub-areas, such as for example a hand, a foot, a tooth region, but also other anatomical structures and/or organs.

The forms of embodiment of the inventive method and of the inventive imaging apparatus described here are to be understood as being examples. Unless explained otherwise in detail, individual forms of embodiment are basically able to be expanded by features of other forms of embodiment. In particular the order of the method steps of the inventive method is to be understood as an example. The individual steps can also be carried out in another order or can overlap in time with each other partly or completely. For example the steps of acquisition of the information about the region of the body and the carrying out of the first imaging examination can take place in any given order after one another or at least partly overlapping. Equally, the provision of the image data and of the input option for adaptation of the parameter of the imaging area as well as the provision of the help for adaptation of the imaging area as a function of the information about the region of the body can be carried out in any given order after one another or at least partly overlapping.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A computer-implemented method for determining an imaging protocol for acquiring image data of a diagnostically relevant sub-area of a region of a body of a patient via an imaging apparatus, the computer-implemented method comprising:
acquiring information about the region of the body;
provisioning an input option for acquiring information about a sub-area of the region of the body as a function of the information about the region of the body, wherein the input option includes a plurality of sub-areas of the region of the body;
acquiring the diagnostically relevant sub-area as a function of the input option for acquiring the information about the sub-area of the region of the body;
determining the imaging protocol for acquiring the image data of the diagnostically relevant sub-area of the region of the body of the patient as a function of the diagnostically relevant sub-area; and
provisioning the imaging protocol for acquiring the image data of the diagnostically relevant sub-area of the region of the body via the imaging apparatus.

2. The computer-implemented method as claimed in claim 1, wherein the acquiring of the information about the region of the body comprises:
acquiring at least one of patient data, image data from previous examinations or an examination instruction.

3. The computer-implemented method as claimed in claim 1, wherein the determining of the imaging protocol for acquiring the image data of the diagnostically relevant sub-area of the region of the body of the patient as a function of the diagnostically relevant sub-area comprises:
selecting an imaging protocol from a plurality of imaging protocols stored in a database.

4. The computer-implemented method as claimed in claim 1, further comprising:
determining an imaging parameter of the imaging protocol.

5. The computer-implemented method as claimed in claim 1, wherein the acquiring of the diagnostically relevant sub-area as a function of the input option for acquiring the information about the sub-area of the region of the body comprises:
acquiring a plurality of diagnostically relevant sub-areas, wherein
the determining of the imaging protocol for acquiring the image data of the diagnostically relevant sub-area of the region of the body of the patient as a function of the diagnostically relevant sub-area includes establishing an imaging protocol for each of the plurality of diagnostically relevant sub-areas, and
the provisioning of the imaging protocol for acquiring the image data of the diagnostically relevant sub-area of the region of the body via the imaging apparatus includes provisioning an imaging protocol for each of the plurality of diagnostically relevant sub-areas.

6. The computer-implemented method as claimed in claim 1, wherein the acquiring of the diagnostically relevant sub-area as a function of the input option for acquiring the information about the sub-area of the region of the body comprises:
acquiring a plurality of diagnostically relevant sub-areas, wherein
the computer-implemented method further includes determining a common imaging area of the region of the body with the aid of the plurality of diagnostically relevant sub-areas, and
the determining of the imaging protocol for acquiring the image data of the diagnostically relevant sub-areas of the region of the body of the patient is undertaken as a function of the common imaging area.

7. The computer-implemented method as claimed in claim 1, further comprising:
provisioning of a further input option for acquiring further information about the diagnostically relevant sub-area; and
acquiring an input as a function of the further input option for acquiring the further information about the diagnostically relevant sub-area, wherein
the determining of the imaging protocol for acquiring the image data of the diagnostically relevant sub-area of the region of the body of the patient is additionally undertaken as a function of the further information.

8. The computer-implemented method as claimed in claim 7, wherein the further input option for acquiring the further information about the diagnostically relevant sub-area includes at least one of an imaging parameter of the imaging protocol, a number of imaging examinations, an order of imaging examinations, or a partial area of the sub-area of the region of the body of the patient.

9. The computer-implemented method as claimed in claim 7, wherein the provisioning of the further input option for acquiring the further information about the diagnostically relevant sub-area comprises:
establishing a provisional imaging area, wherein
the provisional imaging area is adapted to the diagnostically relevant sub-area.

10. The computer-implemented method as claimed in claim 1, wherein the provisioning of the input option for acquiring the information about the sub-area of the region of the body as a function of the information about the region of the body comprises:
provisioning at least one of a graphical representation of the region of the body, a graphical selection of the sub-area of the region of the body, or a text-based selection of the sub-area of the region of the body.

11. The computer-implemented method as claimed in claim 10, wherein at least one of the graphical representation or the graphical selection includes a two-dimensional representation of at least one of the region of the body or of the sub-area of the region of the body.

12. The computer-implemented method as claimed in claim 1, wherein the region of the body of the patient comprises at least one of a jaw region or tooth region.

13. An imaging apparatus for acquiring image data of a diagnostically relevant sub-area of a region of a body of a patient, the imaging apparatus comprising:
a control unit configured to carry out, via the imaging apparatus, a method as claimed in claim 1;
an output unit configured to provide, to a user of the imaging apparatus, the input option for acquiring the information about the sub-area of the region of the body; and
a user interface configured to establish and provide the imaging protocol as a function of the diagnostically relevant sub-area.

14. The imaging apparatus as claimed in claim 13, wherein the imaging apparatus is a magnetic resonance apparatus.

15. A non-transitory computer-readable storage medium storing computer-executable instructions that, when executed by a processing unit of an imaging apparatus, cause the imaging apparatus to carry out the computer-implemented method as claimed in claim 1.

16. The computer-implemented method as claimed in claim 2, wherein the determining of the imaging protocol for acquiring the image data of the diagnostically relevant sub-area of the region of the body of the patient as a function of the diagnostically relevant sub-area comprises:
selecting an imaging protocol from a plurality of imaging protocols stored in a database.

17. The computer-implemented method as claimed in claim 3, further comprising:
determining an imaging parameter of the imaging protocol.

18. The computer-implemented method as claimed in claim 3, wherein the acquiring of the diagnostically relevant sub-area as a function of the input option for acquiring the information about the sub-area of the region of the body comprises:
acquiring a plurality of diagnostically relevant sub-areas, wherein
the determining of the imaging protocol for acquiring the image data of the diagnostically relevant sub-area of the region of the body of the patient as a function of the diagnostically relevant sub-area includes establishing an imaging protocol for each of the plurality of diagnostically relevant sub-areas, and
the provisioning of the imaging protocol for acquiring the image data of the diagnostically relevant sub-area of the region of the body via the imaging apparatus includes provisioning an imaging protocol for each of the plurality of diagnostically relevant sub-areas.

19. The computer-implemented method as claimed in claim 3, wherein the acquiring of the diagnostically relevant sub-area as a function of the input option for acquiring the information about the sub-area of the region of the body comprises:
acquiring a plurality of diagnostically relevant sub-areas, wherein
the computer-implemented method further includes determining a common imaging area of the region of the body with the aid of the plurality of diagnostically relevant sub-areas, and
the determining of the imaging protocol for acquiring the image data of the diagnostically relevant sub-areas of the region of the body of the patient is undertaken as a function of the common imaging area.

20. The computer-implemented method as claimed in claim 3, further comprising:
provisioning of a further input option for acquiring further information about the diagnostically relevant sub-area; and
acquiring an input as a function of the further input option for acquiring the further information about the diagnostically relevant sub-area, wherein
the determining of the imaging protocol for acquiring the image data of the diagnostically relevant sub-area of the region of the body of the patient is additionally undertaken as a function of the further information.
